(19) United States
(12) Patent Application Publication
(10) Pub. No.: US 2025/0213813 A1
(43) Pub. Date: Jul. 3, 2025

(54) HYDROGEN-OXYGEN MIXED GAS INHALATION DEVICE

(71) Applicant: HELIX JAPAN CO., LTD., Tokyo (JP)

(72) Inventors: Takaaki ARISAWA, Chiyoda-ku, Tokyo (JP); Shiro EHARA, Tokyo (JP)

(21) Appl. No.: 18/851,203

(22) PCT Filed: Feb. 20, 2023
(86) PCT No.: PCT/JP2023/005880

§ 371 Date: Sep. 26, 2024

(30) Foreign Application Priority Data
Apr. 5, 2022 (JP) 2022-063164

(51) Int. Cl. A61M 16/12; A61K 33/00; A61M 16/00; A63B 71/04

(52) U.S. Cl. CPC A61M 16/122; A61K 33/00; A61M 16/0063; A63B 71/04; A61M 2202/0208; A61M 2205/3334; A63B 2208/053; A63B 2213/005; A63B 2220/56

(57) ABSTRACT

The invention provides a hydrogen-oxygen mixed gas inhalation device for improving the health condition of a user. The hydrogen-oxygen mixed gas inhalation device comprises a hydrogen supply unit, an oxygen supply unit, an air supply unit, a mixer connected to the hydrogen supply unit, the oxygen supply unit, and the air supply unit, a first compressor connected to the mixer, a flow controller connected to the first compressor, and a pressure vessel connected to the flow controller. The pressure vessel maintains an internal pressure higher than 1 atmosphere, and the user inhales the hydrogen-oxygen mixed gas with a necessary and sufficient flow rate inside the pressure vessel.

### TECHNICAL FIELD

The present invention relates to a hydrogen-oxygen mixed gas inhalation device.

### BACKGROUND ART

Conventionally, devices referred to as “oxygen capsules” or “oxygen boxes” have been used with the purpose of providing users with fatigue recovery or relaxation effects. Typically, these devices house the user in a sealed capsule, pump air with an increased oxygen concentration into it, and raise the internal pressure to efficiently allow the user's body to absorb oxygen.

For example, Patent Document 1 proposes an oxygen capsule, which, in contrast to the common style where one user lies inside the capsule, is large enough to accommodate several people sitting and is structured to be divided for transportation.

### PRIOR ART DOCUMENTS

### Patent Documents

Patent Document 1: JP 2018-20097 A

### SUMMARY OF THE INVENTION

### Problem to be Solved by the Invention

However, while inhaling air with an increased oxygen concentration has various benefits, there is also the drawback that reactive oxygen species (hydroxyl radicals), which are harmful to health, increase in the body. Moreover, the larger the size of the oxygen capsule, the more difficult it becomes to sufficiently raise the internal pressure without using medical oxygen cylinders.

Therefore, the present invention aims to provide various health-improving effects of hydrogen itself and, in addition, the effect of reducing reactive oxygen species by having the user inhale a mixed gas of hydrogen, oxygen, and air (hereinafter referred to as “hydrogen-oxygen mixed gas”) at a necessary and sufficient flow rate. Furthermore, the invention seeks to enhance the absorption efficiency of the hydrogen-oxygen mixed gas into the body by generating sufficient internal pressure within a pressure vessel.

### Means for Solving the Problem

A hydrogen-oxygen mixed gas inhalation device according to the first invention to solve the above problem comprises a hydrogen supply unit, an oxygen supply unit, an air supply unit, a mixer connected to the hydrogen supply unit, the oxygen supply unit, and the air supply unit, a first compressor connected to the mixer, a flow controller connected to the first compressor, a pressure vessel connected to the flow controller, wherein the hydrogen supply unit supplies hydrogen gas to the mixer, the oxygen supply unit supplies oxygen gas to the mixer, the air supply unit supplies flow-regulated air to the mixer, the mixer supplies a first mixed gas, which is a mixture of the hydrogen gas supplied from the hydrogen supply unit, the oxygen gas supplied from the oxygen supply unit, and the air supplied from the air supply unit, to the first compressor, the first compressor supplies a second mixed gas, which is the compressed first mixed gas supplied from the mixer, to the flow controller, the flow controller supplies a third mixed gas, which is the flow-regulated second mixed gas supplied from the first compressor, to the pressure vessel, the pressure vessel maintains an internal pressure higher than 1 atmosphere, and the user inhales the third mixed gas inside the pressure vessel.

By using such a device and inhaling the hydrogen-oxygen mixed gas at a necessary and sufficient flow rate, the user can obtain the various health-improving effects of hydrogen itself, in addition to the effect of reducing reactive oxygen species by hydrogen.

A hydrogen-oxygen mixed gas inhalation device according to the second invention to solve the above problem is the hydrogen-oxygen mixed gas inhalation device according to the first invention, further comprising a second compressor connected to the pressure vessel and an inhaler connected to the flow controller, wherein the second compressor supplies compressed air into the pressure vessel, and the user inhales the third mixed gas by attaching the inhaler to their nose or mouth.

By using such a device, sufficient internal pressure can be generated in the pressure vessel using compressed air supplied externally, separate from the hydrogen-oxygen mixed gas inhaled by the user, allowing the user to enhance the absorption efficiency of the hydrogen-oxygen mixed gas into the body even in a relatively large pressure vessel.

A hydrogen-oxygen mixed gas inhalation device according to the third invention to solve the above problem is the hydrogen-oxygen mixed gas inhalation device according to the first or second invention, further comprising a pressure sensor installed in the pressure vessel, wherein the pressure sensor detects changes in the internal pressure of the pressure vessel and transmits the information to the flow controller, and the flow controller automatically adjusts the pressure at which the third mixed gas is discharged in response to changes in the internal pressure of the pressure vessel to maintain a constant flow rate of the third mixed gas.

By using such a device, the hydrogen-oxygen mixed gas is always automatically adjusted to a necessary and sufficient flow rate, allowing the user to inhale the hydrogen-oxygen mixed gas more safely, easily, and economically.

A hydrogen-oxygen mixed gas inhalation device according to the fourth invention to solve the above problem is the hydrogen-oxygen mixed gas inhalation device according to the third invention, wherein the pressure vessel is formed by connecting two or more pressure vessel units.

By using such a device, it becomes possible to flexibly install a hydrogen-oxygen mixed gas inhalation device of optimal size according to the size of the installation site by connecting any number of pressure vessel units.

A muscle strengthening method according to the fifth invention to solve the above problem is a method for enhancing muscle strength to improve human motor function, comprising a step of performing strength training in an environment where a low oxygen state is maintained, and a step of using the hydrogen-oxygen mixed gas inhalation device according to the first invention.

By using such a method, the user can strengthen muscles more quickly and efficiently than by performing muscle training in a normal atmospheric environment.

### Effects of the Invention

According to the present invention, the user can obtain various health-improving effects of hydrogen itself and the effect of reducing reactive oxygen species by hydrogen, in addition to enhancing the absorption efficiency of the hydrogen-oxygen mixed gas into the body by generating sufficient internal pressure in the pressure vessel. Furthermore, it becomes possible to strengthen muscles more quickly and efficiently.

### BRIEF DESCRIPTION OF THE DRAWINGS

FIG. 1 is a diagram showing the structure of the first embodiment of the hydrogen-oxygen mixed gas inhalation device according to the present invention.

FIG. 2 is a graph showing the effect of reducing reactive oxygen species by hydrogen.

FIG. 3 is a diagram showing the structure of the second embodiment of the hydrogen-oxygen mixed gas inhalation device according to the present invention.

FIG. 4 is a diagram showing the structure of the third embodiment of the hydrogen-oxygen mixed gas inhalation device according to the present invention.

FIG. 5 is an external view of the fourth embodiment of the hydrogen-oxygen mixed gas inhalation device according to the present invention.

FIG. 6 is a diagram showing the procedure of the muscle strengthening method, which is the fifth embodiment of the hydrogen-oxygen mixed gas inhalation device according to the present invention.

### MODE FOR CARRYING OUT THE INVENTION

Hereinafter, the mode for carrying out the present invention will be described with reference to the drawings.

### First Embodiment

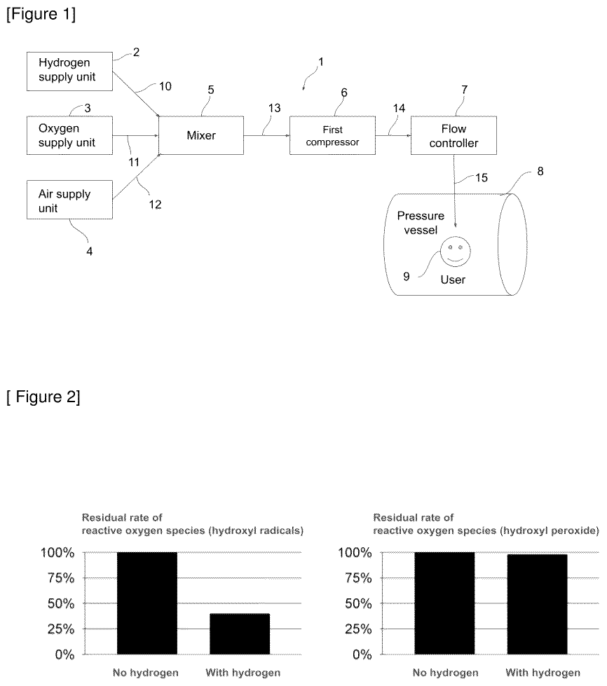

FIG. 1 is a diagram showing the structure of the first embodiment of the hydrogen-oxygen mixed gas inhalation device according to the present invention. The hydrogen-oxygen mixed gas inhalation device according to the present invention is used for the purpose of improving the health condition of the user. In the present invention, “user” refers to a human, but in the first embodiment, it is not necessarily limited to humans and also includes small animals such as pets.

As shown in FIG. 1, the hydrogen-oxygen mixed gas inhalation device 1 according to the present invention includes a hydrogen supplier 2, an oxygen supplier 3, an air supplier 4, a mixer 5, a first compressor 6, a flow controller 7, and a pressure vessel 8.

The hydrogen supplier 2 mainly uses a hydrogen generator that employs a method of electrolytic water decomposition. It is desirable that the hydrogen generator has a hydrogen gas supply capacity of at least 1 liter per minute. Additionally, instead of such a hydrogen generator, a medical hydrogen cylinder or the like may be used.

The oxygen supplier 3 mainly uses an oxygen concentrator that employs a method of increasing oxygen concentration by adsorbing nitrogen from the air using zeolite. It is desirable that the oxygen concentrator has an oxygen gas supply capacity of at least 5 liters per minute with an oxygen concentration of 70% or more. Additionally, instead of such an oxygen concentrator, a medical oxygen cylinder or the like may be used.

The air supplier 4 uses a device that allows adjustment of the airflow rate by mechanisms such as a carburetor.

The hydrogen supplier 2, oxygen supplier 3, and air supplier 4 are each connected to the mixer 5. The mixer 5 generates hydrogen-oxygen mixed gas (hereinafter referred to as “first mixed gas”) 13 by mixing hydrogen gas 10 supplied from the hydrogen supplier 2, oxygen gas 11 supplied from the oxygen supplier 3, and air 12 supplied from the air supplier 4.

At this time, by adjusting the airflow rate of the air 12 supplied from the air supplier 4, the mixed gas of hydrogen gas 10 and oxygen gas 11 is diluted to generate a safe first mixed gas 13 with a hydrogen concentration that does not reach the explosive limit. The airflow rate adjustment of the air by the air supplier 4 can be done manually, but it may also be done automatically in conjunction with a hydrogen concentration sensor installed in the mixer 5.

The mixer 5 supplies the generated first mixed gas 13 to the first compressor 6. The first compressor 6 compresses the first mixed gas 13 supplied from the mixer 5 and supplies the second mixed gas 14, which has an increased pressure, to the flow controller 7. The first compressor 6 mainly uses an oil-free compressor to prevent contamination of the second mixed gas 14 by oil mist, but other types of compressors may be used as long as they can keep the second mixed gas clean.

The flow controller 7 adjusts the flow rate of the second mixed gas 14 supplied from the first compressor 6 to an appropriate value and supplies the third mixed gas 15 to the pressure vessel 8.

The reason for supplying the third mixed gas to the pressure vessel 8 via the first compressor 6 and the flow controller 7, rather than directly supplying the first mixed gas 13 generated by the mixer 5, is as follows.

Namely, the airflow rate necessary and sufficient for a single breath of a human is about 10 liters per minute. However, when using a hydrogen generator employing the method of electrolytic water decomposition as the hydrogen supplier 2 and an oxygen concentrator employing the method of increasing oxygen concentration by adsorbing nitrogen from the air using zeolite as the oxygen supplier 3, the pressure of the hydrogen gas 10 and oxygen gas 11 supplied from both devices is not sufficient to ensure a flow rate of approximately 10 liters per minute for the first mixed gas 13 generated by the mixer 5.

Therefore, it is necessary to compress the first mixed gas 13 by the first compressor 6 to create the second mixed gas 14 with a flow rate increased to over 10 liters per minute, and further adjust the flow rate of the second mixed gas 14 using the flow controller 7 so that it can always be adjusted to the appropriate value of about 10 liters per minute regardless of the internal pressure of the pressure vessel.

Accordingly, when using a medical hydrogen cylinder as the hydrogen supplier 2 and a medical oxygen cylinder as the oxygen supplier 3, the first mixed gas 13 will have a sufficiently high flow rate, so it is unnecessary to further increase the pressure of the first mixed gas 13 by the first compressor 6. However, even in this case, it is necessary to adjust the flow rate of the first mixed gas 13 using the flow controller 7 because supplying an excessive amount of hydrogen and oxygen beyond the airflow rate that a human can breathe would be economically wasteful.

The pressure vessel 8 is a sealed container that maintains an internal pressure higher than 1 atmosphere. The user 9 inhales the third mixed gas supplied from the flow controller 7 within this pressure vessel 8 at a necessary and sufficient flow rate.

The reason for maintaining a high internal pressure in the pressure vessel 8 is that the arterial blood oxygen partial pressure of a human increases as the atmospheric pressure surrounding the human increases. That is, in the medical field, the following formula has been empirically confirmed:

$$Arterial blood oxygen partial pressure ( mm Hg ) = 760 - 47 ) \times 0.21 - 40 / 0.8$$

In the above equation, the arterial blood oxygen partial pressure on the left side increases as the right side 760 mmHg (1 atmosphere) increases. Thus, in a high-pressure environment, oxygen absorption into the body is more efficiently performed. The same relationship is presumed to hold true for hydrogen.

If the internal pressure of the pressure vessel 8 is maintained at 2 atmospheres or higher, the hydrogen-oxygen mixed gas inhalation device 1 according to the present invention will be subject to various regulations to ensure safety as a medical device. Therefore, the internal pressure of the pressure vessel 8 is most economically set at 1.9 atmospheres. Of course, the internal pressure may be set at 2 atmospheres or more if greater health improvement effects are prioritized.

The method of increasing the internal pressure of the pressure vessel 8, in the first embodiment, is by supplying the third mixed gas 15. According to this method, when using a hydrogen generator employing the method of electrolytic water decomposition as the hydrogen supplier 2 and an oxygen concentrator employing the method of increasing oxygen concentration by adsorbing nitrogen from the air using zeolite as the oxygen supplier 3, the size of the pressure vessel 8 will be relatively small for one human or a small animal to maintain sufficient internal pressure.

In other words, the smaller the volume of the pressure vessel, the higher the internal pressure can be maintained. Therefore, when using the hydrogen-oxygen mixed gas inhalation device according to the present invention for small animals such as pets, it is efficient to use a small capsule as the pressure vessel 8 exclusively for small animals.

Next, the main effects of inhaling hydrogen gas in addition to oxygen gas in the hydrogen-oxygen mixed gas inhalation device according to the present invention will be described.

Inhaling air with an increased oxygen concentration has various benefits, but it also has the disadvantage of increasing harmful reactive oxygen species (hydroxyl radicals) in the body, which are harmful to health. Hydrogen has the effect of selectively reducing only harmful reactive oxygen species.

Namely, reactive oxygen species include harmful hydroxyl radicals, which cause arteriosclerosis and genetic damage, and beneficial substances like hydrogen peroxide, which fight bacteria and viruses. It has been recognized that hydrogen absorbed in the body acts only on these harmful hydroxyl radicals, eliminating them, and has little effect on beneficial substances like hydrogen peroxide.

FIG. 2 is a graph showing the effect of hydrogen in reducing reactive oxygen species. As shown in FIG. 2, hydrogen absorbed in the body reduces harmful hydroxyl radicals by about 40%, while it has almost no effect on beneficial hydrogen peroxide. This stands in sharp contrast to vitamin C, which also has the effect of reducing reactive oxygen species but acts on both harmful hydroxyl radicals and beneficial hydrogen peroxide.

The first embodiment of the hydrogen-oxygen mixed gas inhalation device according to the present invention has been described above. By using such a device and inhaling hydrogen-oxygen mixed gas at a necessary and sufficient flow rate, the user can obtain not only the various health improvement effects inherent to hydrogen but also the effect of reducing reactive oxygen species by hydrogen.

### Second Embodiment

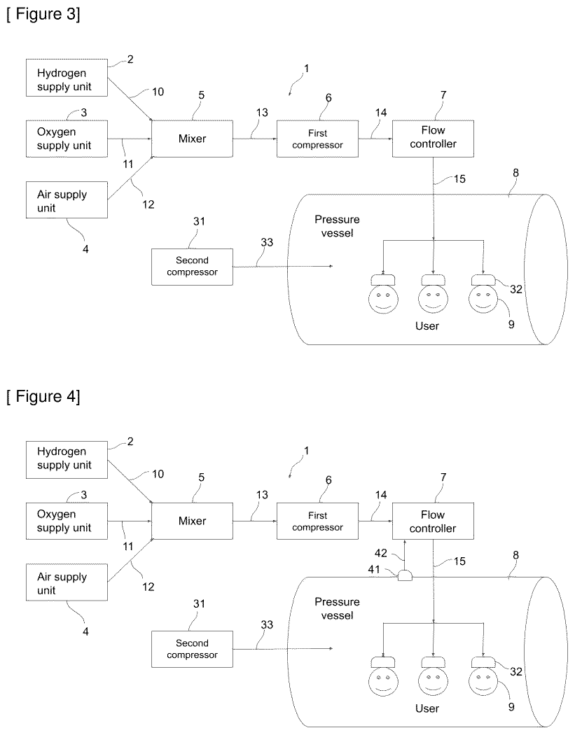

FIG. 3 is a diagram showing the structure of a second embodiment of the hydrogen-oxygen mixed gas inhalation device according to the present invention. As shown in FIG. 3, the hydrogen-oxygen mixed gas inhalation device 1 according to the present invention includes a hydrogen supply unit 2, an oxygen supply unit 3, an air supply unit 4, a mixer 5, a first compressor 6, a flow controller 7, a pressure vessel 8, a second compressor 31, and an inhaler 32. That is, the second embodiment is an addition of the second compressor 31 and the inhaler 32 to the first embodiment.

In the second embodiment, the second compressor 31 supplies compressed air 33 to the pressure vessel 8. By this method, compared to the pressurization method of the first embodiment, where the internal pressure of the pressure vessel 8 is increased by supplying the third mixed gas 15, it becomes possible to more easily increase the internal pressure of the pressure vessel 8. As a result, even in a large pressure vessel 8 that can accommodate multiple users 9, it is possible to achieve a sufficiently high-pressure environment inside.

On the other hand, according to this pressurization method using the second compressor, the internal pressure of the pressure vessel 8 is increased by compressed air. Therefore, in this state, the third mixed gas 15 supplied from the flow controller 7 to the pressure vessel 8 would be diluted inside the pressure vessel, and the users 9 would not be able to inhale the necessary and sufficient concentrations of hydrogen and oxygen.

Therefore, in the second embodiment, the user 9 attaches the inhaler 32 connected to the flow controller 7 to their nose or mouth, and inhales the undiluted third mixed gas 15 directly. The inhaler 32 used here is suitable as an inhalation mask covering the user's nose and mouth or a cannula inserted into the nasal cavity, but other devices that can directly inhale the third mixed gas 15 may also be used.

The above is the second embodiment of the hydrogen-oxygen mixed gas inhalation device according to the present invention. By using such a device, and by generating sufficient internal pressure in the pressure vessel using compressed air 33 supplied externally, separately from the third mixed gas 15, the user 9 can improve the absorption efficiency of hydrogen and oxygen contained in the third mixed gas 15 into the body, even in a relatively large pressure vessel 8.

### Third Embodiment

FIG. 4 is a diagram showing the structure of a third embodiment of the hydrogen-oxygen mixed gas inhalation device according to the present invention. As shown in FIG. 4, the hydrogen-oxygen mixed gas inhalation device 1 according to the present invention includes a hydrogen supply unit 2, an oxygen supply unit 3, an air supply unit 4, a mixer 5, a first compressor 6, a flow controller 7, a pressure vessel 8, a second compressor 31, an inhaler 32, and a pressure sensor 41. That is, the third embodiment is an addition of a pressure sensor 41 to the second embodiment.

In the third embodiment, the pressure sensor 41 is installed in the pressure vessel 8, detects changes in the internal pressure of the pressure vessel 8, and transmits that pressure information 42 to the flow controller 7 without delay. The flow controller 7 analyzes the received pressure information 42 and adjusts the pressure at which the third mixed gas 15 is discharged according to changes in the internal pressure of the pressure vessel 8.

That is, when the internal pressure of the pressure vessel 8 increases due to the compressed air 33 supplied from the second compressor, in order to maintain the flow rate of the third mixed gas 15 at an appropriate value of about 10 liters per minute, the discharge pressure of the third mixed gas 15 must be increased in response to the rise in internal pressure of the pressure vessel 8.

In the third embodiment, by applying the necessary program to the flow controller 7 so that this flow rate adjustment is performed automatically rather than manually, the flow rate of the third mixed gas 15 that the user 9 inhales through the inhaler 32 can always be maintained at a necessary and sufficient appropriate value. For such a flow controller 7, for example, a proportional solenoid valve can be used.

The above is the third embodiment of the hydrogen-oxygen mixed gas inhalation device according to the present invention. By using such a device, the user 9 can more safely, easily, and economically absorb hydrogen-oxygen mixed gas into the body by automatically adjusting the flow rate of the inhaled third mixed gas 15 to always be at a necessary and sufficient appropriate value.

### Fourth Embodiment

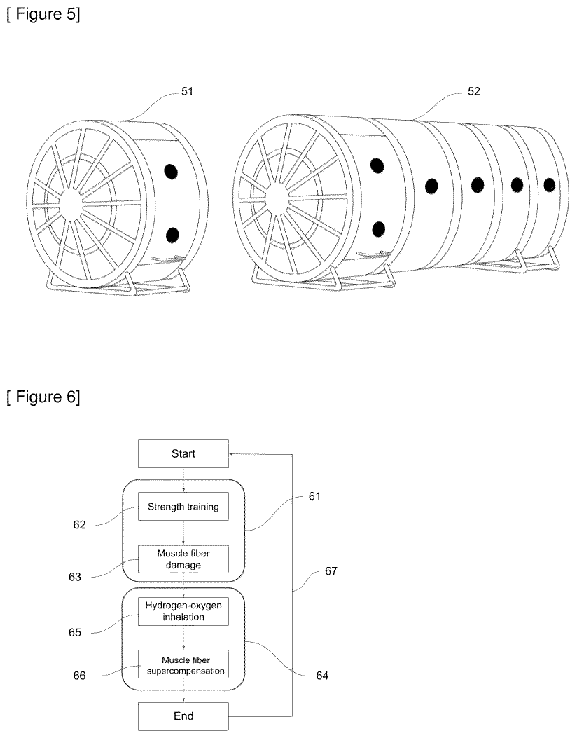

FIG. 5 is a diagram showing the exterior of a fourth embodiment of the hydrogen-oxygen mixed gas inhalation device according to the present invention. As shown in FIG. 5, in the fourth embodiment, by making it possible to assemble a connected pressure vessel 52 using any number of pressure vessel units 51, it becomes possible to flexibly install a hydrogen-oxygen mixed gas inhalation device of the optimal size according to the available installation space.

### Fifth Embodiment

FIG. 6 is a diagram showing the procedure of the muscle strengthening method, which is the fifth embodiment of the hydrogen-oxygen mixed gas inhalation device according to the present invention. This muscle strengthening method aims to improve human motor functions.

As shown in FIG. 6, the user performs muscle training 62 under an environment 61 where a low oxygen state is maintained. While the oxygen concentration in the atmosphere is about 21%, it is desirable that the oxygen concentration in the environment where this muscle training 62 is performed is maintained at about 14%, equivalent to high-altitude training at an altitude of 3,000 meters, but in the case of elderly people or beginners, it may be relaxed to about 18% from a safety perspective.

Moreover, the muscle training 62 is preferably anaerobic exercise aimed at training fast-twitch muscles. This is because the effect of the supercompensation 66 of muscle fibers, described later, is more apparent in fast-twitch muscles. Through such muscle training 62, the user can efficiently achieve muscle fiber damage 63.

After performing such muscle training 62 and achieving muscle fiber damage 63, the user uses the hydrogen-oxygen mixed gas inhalation device 64 according to the present invention. That is, by efficiently inhaling a necessary and sufficient amount of hydrogen-oxygen mixed gas 65 under the high-pressure environment inside the pressure vessel that constitutes the hydrogen-oxygen mixed gas inhalation device 64, the supercompensation 66 of muscle fibers is promoted.

By repeating this procedure 67, the user can efficiently strengthen muscles in a shorter period than by performing muscle training under normal atmospheric conditions. The above is the muscle strengthening method, which is the fifth embodiment of the hydrogen-oxygen mixed gas inhalation device according to the present invention.

The first to fifth embodiments described above are examples for explaining the present invention, and the present invention is not limited to these embodiments. The present invention can be implemented in various forms without departing from the gist of the invention.

### INDUSTRIAL APPLICABILITY

The present invention makes it possible to efficiently improve the health conditions and motor functions of humans and pets. As the super-aging society rapidly progresses not only in Japan but also in Western and Asian countries, the need for efficient improvement of health conditions and motor functions is expected to increase. Therefore, the present invention has industrial applicability.

### DESCRIPTION OF SYMBOLS

1. Hydrogen-oxygen mixed gas inhalation device
2. Hydrogen supply unit
3. Oxygen supply unit
4. Air supply unit
5. Mixer
6. First compressor
7. Flow controller
8. Pressure vessel
9. User
10. Hydrogen gas
11. Oxygen gas
12. Air
13. First mixed gas
14. Second mixed gas
15. Third mixed gas
31. Second compressor
32. Inhaler
33. Compressed air
41. Pressure sensor
42. Pressure information
51. Pressure vessel unit
52. Linked pressure vessel
61. Low oxygen environment
62. Strength training
63. Muscle fiber damage
64. Hydrogen-oxygen mixed gas inhalation device
65. Hydrogen-oxygen inhalation
66. Muscle fiber supercompensation
67. Repetition of procedure

1. A hydrogen-oxygen mixed gas inhalation device for improving health condition of a user, comprising:
a hydrogen supply unit;
an oxygen supply unit;
an air supply unit;
a mixer connected to the hydrogen supply unit, the oxygen supply unit, and the air supply unit;
a first compressor connected to the mixer;
a flow controller connected to the first compressor; and
a pressure vessel connected to the flow controller,
wherein the hydrogen supply unit supplies hydrogen gas to the mixer,
the oxygen supply unit supplies oxygen gas to the mixer,
the air supply unit supplies flow-regulated air to the mixer,
the mixer supplies a first mixed gas, which is a mixture of the hydrogen gas supplied from the hydrogen supply unit, the oxygen gas supplied from the oxygen supply unit, and the air supplied from the air supply unit, to the first compressor,
the first compressor supplies a second mixed gas, which is the compressed first mixed gas supplied from the mixer, to the flow controller,
the flow controller supplies a third mixed gas, which is the flow-regulated second mixed gas supplied from the first compressor, to the pressure vessel,
the pressure vessel maintains an internal pressure higher than 1 atmosphere, and
the user inhales the third mixed gas inside the pressure vessel.

2. The hydrogen-oxygen mixed gas inhalation device according to claim 1, further comprising:
a second compressor connected to the pressure vessel; and
an inhaler connected to the flow controller,
wherein the second compressor supplies compressed air into the pressure vessel, and
the user inhales the third mixed gas by attaching the inhaler to their nose or mouth.

3. The hydrogen-oxygen mixed gas inhalation device according to claim 1, further comprising:
a pressure sensor installed in the pressure vessel,
wherein the pressure sensor detects changes in the internal pressure of the pressure vessel and transmits the information to the flow controller, and
the flow controller automatically adjusts the pressure at which the third mixed gas is discharged in response to changes in the internal pressure of the pressure vessel to maintain a constant flow rate of the third mixed gas.

4. The hydrogen-oxygen mixed gas inhalation device according to claim 3, wherein the pressure vessel is formed by connecting two or more pressure vessel units.

5. A method for enhancing muscle strength to improve human motor function, comprising:
a step of performing strength training in an environment where a low oxygen state is maintained; and
a step of using the hydrogen-oxygen mixed gas inhalation device according to claim 1.

6. The hydrogen-oxygen mixed gas inhalation device according to claim 2, further comprising:
a pressure sensor installed in the pressure vessel,
wherein the pressure sensor detects changes in the internal pressure of the pressure vessel and transmits the information to the flow controller, and
the flow controller automatically adjusts the pressure at which the third mixed gas is discharged in response to changes in the internal pressure of the pressure vessel to maintain a constant flow rate of the third mixed gas.

7. The hydrogen-oxygen mixed gas inhalation device according to claim 6, wherein the pressure vessel is formed by connecting two or more pressure vessel units.
